(19) United States
(12) Patent Application Publication
(10) Pub. No.: US 2017/0095629 A1
(43) Pub. Date: Apr. 6, 2017

(54) TRACHEOSTOMY TUBE ASSEMBLIES AND INNER CANNULAE

(71) Applicant: SMITHS MEDICAL INTERNATIONAL LIMITED, Kent (GB)

(72) Inventors: Timothy Bateman, Kent (GB); Stephen James Field, Kent (GB); Andrew Thomas Jeffrey, Kent (GB)

(73) Assignee: SMITHS MEDICAL INTERNATIONAL LIMITED, Kent (GB)

(21) Appl. No.: 15/126,010

(22) PCT Filed: Mar. 4, 2015
(86) PCT No.: PCT/GB2015/000076

§ 371 Date: Sep. 14, 2016

(30) Foreign Application Priority Data
Mar. 22, 2014 (GB) 1405219.5

(51) Int. Cl. A61M 16/04

(52) U.S. Cl. CPC A61M 16/0427; A61M 16/0438; A61M 16/0465; A61M 16/0488; A61M 2205/0266

(57) ABSTRACT

A tracheostomy tube assembly includes an outer tube (1) and an inner cannula (20, 120, 220 51, 61) inserted into and removable from the tube. The inner cannula has a constant internal diameter along its length and has at its patient end (22, 122, 222) a seal member (23, 123, 223) extending around the cannula. The seal member may be a separate ring 23 of resilient material attached to the outside of the shaft of the cannula or it may be formed of the material of the shaft (121, 221) itself. Alternatively, the seal member (50, 60) may be of a kind that expands when exposed to an elevated temperature or humidity, and may include a shape memory effect material.

This invention relates to an inner cannula of the kind for a tracheostomy tube assembly.

Tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall corrugated both externally and internally and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,019,753 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting.

It is an object of the present invention to provide an alternative inner cannula and tracheostomy tube assembly.

According to one aspect of the present invention there is provided an inner cannula of the above-specified kind, characterised in that the inner cannula includes a tubular shaft having a constant internal diameter along its length, that the cannula has an annular seal member around the shaft adjacent its patient end, and that the seal member is arranged to extend externally around the shaft and make a sliding sealing fit with the inside of an outer tracheostomy tube within which the inner cannula is inserted.

The seal member may be expansible and may, for example, be of a resilient material such as a foam attached with the shaft. Alternatively, the seal member may be formed of material of the shaft itself and may, for example, be formed by folding back the patient end of the shaft to form a short, rearwardly-extending lip around the outside of the patient end. Alternatively, the seal member may be arranged to expand when exposed to temperature above room temperature. The seal member may include a shape memory material arranged to change from a first state at around room temperature to a second state around body temperature such that the seal member expands radially outwardly on change from the first to the second state when inserted in the trachea. Alternatively, the seal member may be arranged to expand when exposed to humidity above room humidity.

According to another aspect of the present invention there is provided a tracheostomy tube assembly including an outer tracheostomy tube and an inner cannula according to the above one aspect of the present invention inserted within the outer tube and being removable therefrom.

An inner cannula and a tracheostomy tube assembly including an inner cannula both in accordance with the present invention, will now be described, by way of example, with reference to the accompanying drawings, which are not to scale, and in which:

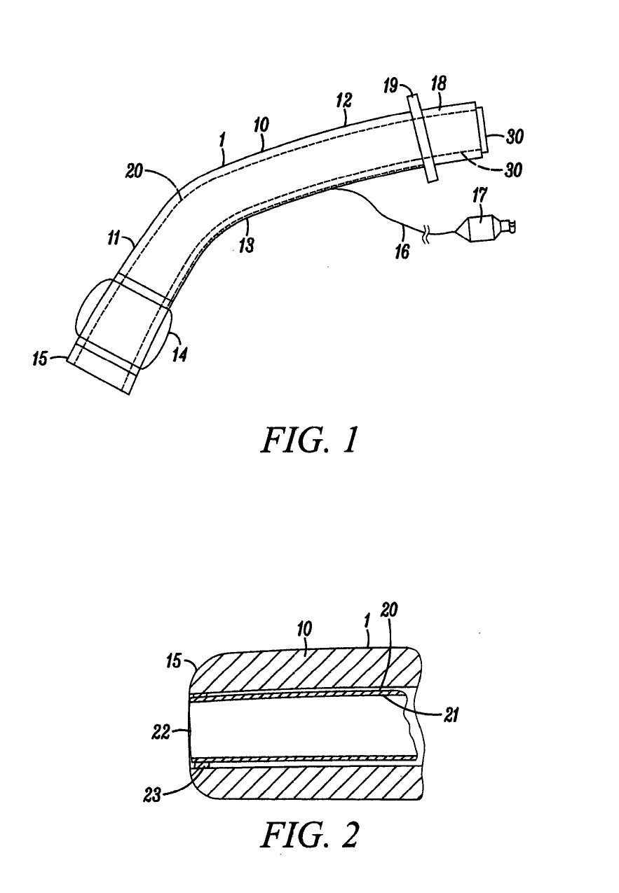

FIG. 1 is a side elevation view of a tracheostomy tube assembly including an inner cannula;

FIG. 2 is an enlarged cross-sectional side elevation of the patient end of the assembly;

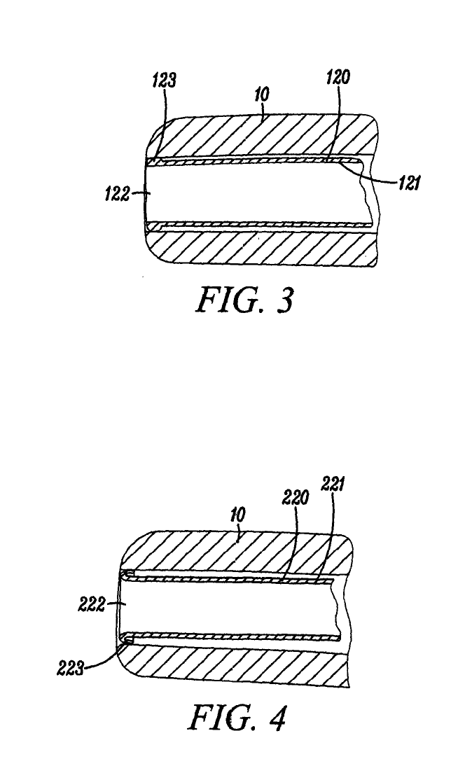

FIGS. 3 and 4 are enlarged cross-sectional side elevation views of the patient end of assemblies with alternative inner cannulae;

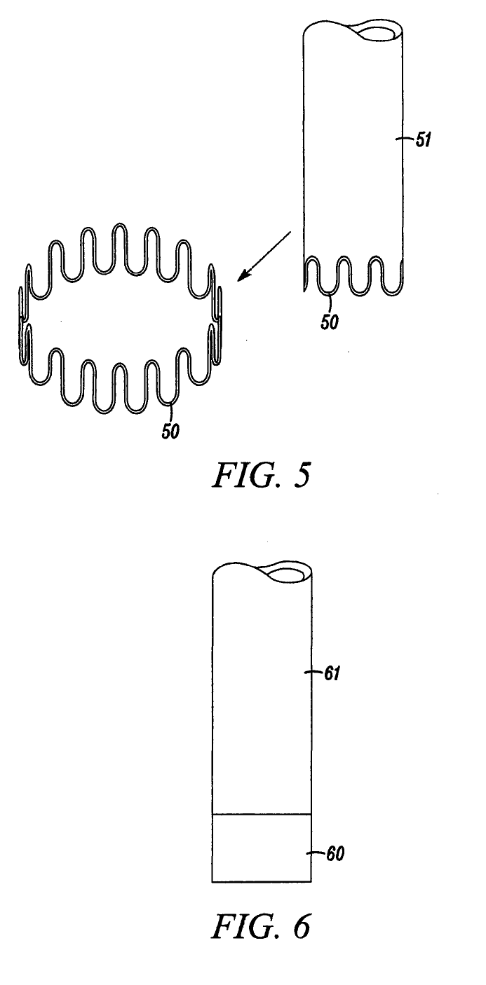

FIG. 5 shows the patient end of an alternative inner cannula; and

FIG. 6 shows the patient end of another alternative inner cannula.

With reference first to FIG. 1, the tracheostomy tube assembly comprises an outer tracheostomy tube 1 and a removable inner cannula 20 inserted within the outer tube. The outer tube 1 has a shaft 10 with a straight forward section 11, a straight rear section 12 and a curved intermediate section 13 linking the forward and rear sections. An inflatable sealing cuff 14 embraces the forward section 11 close to the patient end 15 of the tube, the cuff being inflated via an inflation lumen 16 and a combined connector and inflation indicator 17. At its rear or machine end the outer tube 1 has a hub 18 and flange 19 to which a retaining tape (not shown) can be fastened for securing the tube with the patient's neck. The inside of the hub 18 is formed with keying flats (not shown), of the kind described in EP1938857, adapted to prevent full insertion of an inner cannula of the wrong size. The outer tube 1 could have an internal diameter between about 2 mm and 10 mm, and its length could be between 60mm and 200 mm.

With reference now also to FIG. 2, the inner cannula 20 is formed by a shaft 21 of circular section attached at its rear or machine end with a hub 30. The shaft 21 is of a thin, stiff, flexible plastics material, such as PVC, polyurethane, polyethylene, polypropylene, PTFE or other flexible or semi-rigid plastics material. The external diameter of the shaft 21 along most of its length is selected to be just smaller than the inner diameter of the shaft 10 of the outer tube so that the inner cannula can be readily inserted and removed from the outer tube. The internal diameter of the shaft 21 is constant along its entire length from the hub 30 to the patient end tip 22. An annular seal member 23 is attached to the outside of the shaft 21 at its patient end tip 22 to extend circumferentially around the shaft. The seal member 23 is of an expansible, compressible, resilient foam material. The natural, expanded maximum diameter of the seal member 23 is chosen to be slightly greater than the maximum internal diameter of the shaft 10 of the outer tube 1 allowing for tolerances. In this way, it can be seen that, when the inner cannula 20 is located inside the outer tube 1, the seal member 23 will be in compression between the outside of the inner cannula and the inside of the outer tube. This forms an effective seal between the outside of the inner cannula 20 and the inside of the outer tube 1 at the patient end of the assembly thereby preventing leakage between the inner cannula and the outer tube. This is achieved without altering the inner diameter of the inner cannula 20 at any point along its length. The present arrangement avoids the need for close tolerances in the manufacture of the inner cannula 20 and the outer tube 1, thereby reducing the need for precision manufacture and enabling costs to be kept down. Because contact between the inner cannula 20 and the outer tube 1 is localised at the patient end seal, friction between the inner cannula and the outer tube during insertion and removal is minimised. This can be important because it enables the insertion force to be minimised and thereby reduces the need to stiffen the inner cannula 20 against axial compression forces. This allows its wall to be kept thin and maximises the internal cross section so that resistance to gas flow along the inner cannula is kept low. The reduction of insertion force minimises any axial compression and so ensures accurate alignment of the patient end 22 of the inner cannula 20 and outer tube 1.

The seal member need not be of a foam but could be of a material that expands in use from a first diameter, smaller than the internal diameter of the outer tube, to a second diameter slightly larger than the internal diameter of the outer tube. In this way, the inner cannula can be initially inserted freely within the outer tube and then allowed to expand to seal with the outer tube. Such a seal member could be of a material that swells when exposed to humidity, such as a hydrophilic gel or the like.

The expansible annular sealing member could be provided in other ways, such as shown in FIG. 3 or 4. FIG. 3 shows an inner cannula 120 where the shaft 121 has an integral, moulded annular lip or seal 123 extending around the outside of the patient end 122 of the inner cannula. The annular lip 123 is of the same material as the shaft itself so this may not be resilient but the annular shape of the lip gives it a spring-like characteristic that ensures it is always in an effective sealing relationship with the inside of the outer tube 1.

FIG. 4 shows an alternative inner cannula 220 where the shaft 221 is folded back externally at its patient end 222 around the outside of the cannula by a short distance to form a resilient, spring-like seal in the form of a rearwardly-extending lip 223 that can flex in and out relative to the main body of the shaft 221. In both the arrangements shown in FIGS. 3 and 4 the annular seal 123 or 223 is formed to have a natural maximum external diameter slightly greater than the inner diameter of the outer tube 1, whereas the external diameter of the main part of the shaft 121 or 221 is formed to have an external diameter slightly less than the inner diameter of the outer tube.

The seal could be arranged to expand when the temperature rises from room temperature to close to body temperature. Such a seal could be formed of a closed tube containing a substance that is liquid at room temperature and undergoes a phase transformation to a vapour at temperatures close to body temperature thereby causing the closed tube to expand. Alternatively, as shown in FIG. 5, the seal 50 could be formed by a shape memory material such as of plastics or a metal alloy selected to change from a first to a second phase at a temperature above room temperature and slightly below body temperature. The seal 50 shown in FIG. 5 is in the form of a ring with a wavy or serpentine configuration extending circumferentially around the inner cannula 51 so that the waves on the ring flatten and cause it to expand radially at the higher temperature. The larger diameter of the seal 50 still enables it to be pulled out of the outer tube. When the inner cannula 51 has cooled below the transition temperature it contracts back to its original diameter so that it can be reinserted in the outer tube after cleaning. FIG. 6 shows an inner cannula 61 with a ring 60 of a shape memory plastics material at its tip. Again, this is arranged to expand and contract diametrically at a transition temperature just below body temperature so that it can be inserted freely within the outer tube without the risk of buckling and then expands, as it warms close to body temperature, to form an effective seal with the inside of the outer tube.

1-9. (canceled)

10. An inner cannula for a tracheostomy tube assembly, characterised in that the inner cannula includes a tubular shaft having a constant internal diameter along its length, that the cannula has an annular seal member around the shaft adjacent its patient end, and that the seal member is arranged to extend externally around the shaft and make a sliding sealing fit with the inside of an outer tracheostomy tube within which the inner cannula is inserted.

11. An inner cannula according to claim 10, characterised in that the seal member is expansible.

12. An inner cannula according to claim 11, characterised in that the seal member is of a resilient material such as a foam attached with the shaft.

13. An inner cannula according to claim 10, characterised in that the seal member is formed of material of the shaft itself.

14. An inner cannula according to claim 13, characterised in that the seal member is formed by folding back the patient end of the shaft to form a short, rearwardly-extending lip around the outside of the patient end.

15. An inner cannula according to claim 10, characterised in that the seal member is arranged to expand when exposed to temperature above room temperature.

16. An inner cannula according to claim 15, characterised in that the seal member includes a shape memory material arranged to change from a first state at around room temperature to a second state around body temperature such that the seal member expands radially outwardly on change from the first to the second state when inserted in the trachea.

17. An inner cannula according to claim 11, characterised in that the seal member includes a shape memory material arranged to change from a first state at around room temperature to a second state around body temperature such that the seal member expands radially outwardly on change from the first to the second state when inserted in the trachea.

18. An inner cannula according to claim 10, characterised in that the seal member is arranged to expand when exposed to humidity above room humidity.

19. An inner cannula according to claim 11, characterised in that the seal member is arranged to expand when exposed to humidity above room humidity.

20. A tracheostomy tube assembly including an outer tracheostomy tube and an inner cannula inserted within the outer tube and being removable therefrom, wherein the inner cannula includes a tubular shaft having a constant internal diameter along its length and an annular seal member around the shaft adjacent its patient end, and wherein the seal member is arranged to extend externally around the shaft and make a sliding sealing fit with the inside of the outer tracheostomy tube.
